(19) United States
(12) Patent Application Publication
(10) Pub. No.: US 2023/0347132 A1
(43) Pub. Date: Nov. 2, 2023

(54) METHOD OF PURGING A BLOOD PUMP

(71) Applicant: ABIOMED, Inc., Danvers, MA (US)

(72) Inventors: Scott Corbett, Danvers, MA (US); Soumen Das, Danvers, MA (US); Vladimir Gilman, Danvers, MA (US); Jun Ding, Danvers, MA (US); Jeremy Moretz, Danvers, MA (US)

(73) Assignee: ABIOMED, Inc., Danvers, MA (US)

(21) Appl. No.: 18/111,978

(22) Filed: Feb. 21, 2023

Related U.S. Application Data
(60) Provisional application No. 63/312,277, filed on Feb. 21, 2022.
(60) Provisional application No. 63/398,991, filed on Aug. 18, 2022.

(51) Int. Cl. A61M 60/221; A61M 60/829

(52) U.S. Cl. CPC A61M 60/221; A61M 60/829; A61M 2202/0085

(57) ABSTRACT

Devices, system, and methods are provided based on a technique for purging a blood pump. The technique may include providing a blood pump having an impeller and providing a fluid comprising a bicarbonate. The technique may include flowing the fluid through a first gap between a bearing and an outer surface of a rotatable shaft coupled to the impeller, the bearing and gap being disposed within a lumen of a tubular member and depending on the flow rate of the fluid and the speed of the impeller, the fluid may flow through the first gap and into a second gap between the bearing and a surface of the impeller facing the bearing. The bicarbonate reduces denaturation and adsorption of any blood proteins in the gap(s).

### CROSS-REFERENCE TO RELATED APPLICATIONS

The present application claims priority to U.S. provisional patent applications 63/312,277 Filed Feb. 21, 2022, and 63/398,991 Filed Aug. 18, 2022, each of which is incorporated by reference herein in its entirety.

### TECHNICAL FIELD

The present disclosure relates to a blood pump, in particular an intravascular blood pump, to support a blood flow in a patient's blood vessel and methods for purging such a pump in operation while inserted into a patient.

### BACKGROUND

Blood pumps of different types are known, such as axial blood pumps, centrifugal blood pumps, or mixed-type blood pumps, where the blood flow is caused by both axial and radial forces. One example of a blood pump is the Impella line of blood pumps (e.g., Impella 2.5®, Impella CP®, Impella 5.5®, etc.) which are products of Abiomed of Danvers, Mass. Intravascular blood pumps may be inserted into a patient's vessel such as the aorta by means of a catheter.

In some pump designs, a purge fluid is deployed to keep blood from entering the mechanism and to mitigate the effects of blood on the pump mechanisms. Typically, the purge fluid includes an anticoagulant such as heparin (typically the sodium salt of heparin). The heparin is thought to keep the blood from coagulating in the gap between pump components such as an impeller shaft and the housing. Heparin is a commonly used anticoagulant typically administered in controlled dosages.

In one example, the purge fluid is delivered by a purge cassette that enters a blood pump catheter through a filter assembly and internal purge lumen that carries the purge fluid through the catheter to a purge channel in the motor assembly. The flow of the purge fluid is regulated by an automated controller.

However, such conventional techniques typically lead to denaturation of circulating proteins and biomaterial deposition within the gaps between pump components.

### BRIEF SUMMARY

In various aspects, a blood pump may be provided. The blood pump may include a tubular body having a distal end and a proximal end, and a lumen extending therethrough, the lumen having a central axis. An impeller may be coupled to a flexible shaft disposed at least partially within the lumen. A bearing may be disposed within the lumen, around a portion of the flexible shaft. The bearing may be configured to form a first gap in a radial direction between an outer surface of the flexible shaft and an inner surface of the bearing, and may be configured to form a second gap in an axial direction is between the bearing and a surface of the impeller facing the bearing. The blood pump may be configured to flow a fluid comprising a bicarbonate through the first gap and into the second gap. In some embodiments, the first gap may be 4-9 μm. In some embodiments, the second gap may be 90-110 μm.

In some embodiments, the bicarbonate may be sodium bicarbonate. In some embodiments, the fluid may include 12.5 mEq/L to 100 mEq/L of sodium bicarbonate, such as 25 mEq/L to 50 mEq/L of sodium bicarbonate. In some embodiments, the fluid may include a pharmaceutical therapeutic or prophylactic agent, such as an anticoagulant or a patient-specific pharmaceutical treatment (e.g., a small molecule drug). In some embodiments, the fluid further may include aqueous dextrose.

In some embodiments, a concentration of the fluid comprising the bicarbonate within a volume of space defined by the first gap may be at least 50% of all fluid in the first gap. In some embodiments, the first gap may be filled with the fluid comprising the bicarbonate. In some embodiments, a concentration of the fluid comprising the bicarbonate within a volume of space defined by the second gap may be no more than 40% of all fluid in the second gap.

In various aspects, a system may be provided. The system may include a pump as disclosed herein, and may include a purge bag containing the fluid comprising the bicarbonate, where the purge bag may be operably coupled to the pump. In some embodiments, the first gap may be 4-9 μm. In some embodiments, the second gap may be 90-110 μm.

In some embodiments, the bicarbonate may be sodium bicarbonate. In some embodiments, the fluid may include 12.5 mEq/L to 100 mEq/L of sodium bicarbonate, such as 25 mEq/L to 50 mEq/L of sodium bicarbonate. In some embodiments, the fluid may include a pharmaceutical therapeutic or prophylactic agent, such as an anticoagulant or a patient-specific pharmaceutical treatment (e.g., a small molecule drug). In some embodiments, the fluid further may include aqueous dextrose.

In some embodiments, a concentration of the fluid comprising the bicarbonate within a volume of space defined by the first gap may be at least 50% of all fluid in the first gap. In some embodiments, the first gap may be filled with the fluid comprising the bicarbonate. In some embodiments, a concentration of the fluid comprising the bicarbonate within a volume of space defined by the second gap may be no more than 40% of all fluid in the second gap.

In various aspects, a kit may be provided. The kit may include a pump as disclosed herein. The kit may include a purge bag containing the fluid comprising the bicarbonate. The kit may include a purge cassette configured to couple the purge bag to the pump.

In various aspects, a method for purging a pump may be provided. The method may include providing a pump having an impeller as disclosed herein, and a fluid comprising a bicarbonate. The method may include flowing the fluid through a first gap between a bearing and an outer surface of a rotatable shaft coupled to the impeller, the bearing and gap being disposed within a lumen of a tubular member.

In some embodiments, the bicarbonate may be sodium bicarbonate. In some embodiments, the fluid may include 12.5 mEq/L to 100 mEq/L of sodium bicarbonate, such as 25 mEq/L to 50 mEq/L of sodium bicarbonate. In some embodiments, the fluid may include a pharmaceutical therapeutic or prophylactic agent, such as an anticoagulant or a patient-specific pharmaceutical treatment (e.g., a small molecule drug). In some embodiments, the fluid further may include aqueous dextrose.

In some embodiments, the method may include controlling a flow rate of the fluid such that a concentration of the fluid comprising the bicarbonate within a volume of space defined by the first gap is at least 50% of all fluid in the first gap.

In some embodiments, flowing the fluid through a first gap may include flowing the fluid through a second gap after passing through the first gap, the second gap being between the bearing and a surface of the impeller facing the bearing. In some embodiments, the method may include controlling a flow rate of the fluid such that a concentration of the fluid comprising the bicarbonate within a volume of space defined by the second gap is no more than 40% of all fluid in the second gap.

In some embodiments, the method may include automatically adjusting a flow rate of the fluid based on an operating characteristic of the pump.

In various aspects, a medical device may be provided. The medical device may include a tubular body having a distal end and a proximal end, and a lumen may extend therethrough. The lumen may have a central axis. The medical device may include an outer sheath disposed around the tubular body. The medical device may include a first gap formed in a radial direction between an outer surface of the tubular body and the inner surface of the outer sheath. The medical device may include a second gap formed in an axial direction between the tubular body and the outer sheath. A fluid comprising a bicarbonate may be configured to flow through the first and second gaps to purge the first and second gaps. In some embodiments, the first gap may be 4-9 μm. In some embodiments, the second gap may be 90-110 μm.

In some embodiments, the bicarbonate may be sodium bicarbonate. In some embodiments, the fluid may include 12.5 mEq/L to 100 mEq/L of sodium bicarbonate, such as 25 mEq/L to 50 mEq/L of sodium bicarbonate. In some embodiments, the fluid may include a pharmaceutical therapeutic or prophylactic agent, such as an anticoagulant or a patient-specific pharmaceutical treatment (e.g., a small molecule drug). In some embodiments, the fluid further may include aqueous dextrose.

In some embodiments, a concentration of the fluid comprising the bicarbonate within a volume of space defined by the first gap may be at least 50% of all fluid in the first gap. In some embodiments, the first gap may be filled with the fluid comprising the bicarbonate. In some embodiments, a concentration of the fluid comprising the bicarbonate within a volume of space defined by the second gap may be no more than 40% of all fluid in the second gap.

### BRIEF DESCRIPTION OF DRAWINGS

Hereinafter, the invention will be explained by way of example with reference to the accompanying drawings. The accompanying drawings are not intended to be drawn to scale. In the drawings, each identical or nearly identical component that is illustrated in various figures is represented by a like numeral. For purposes of clarity, not every component may be labelled in every drawing. In the drawings:

FIG. 1 illustrates the blood flow and the purge flow through the gap between the shaft and the housing in the pump;

FIG. 2 is a schematic representation of an intravascular blood pump inserted before the left ventricle, with its inflow cannula positioned in the left ventricle;

FIG. 3 is a schematic longitudinal cross-section of an exemplary prior art blood pump;

FIG. 4 is an enlarged representation of a part of the blood pump of FIG. 3 according to a second embodiment;

FIG. 5 is an enlarged representation of a part of a blood pump according to other embodiments;

FIGS. 6A and 6B illustrates bicarbonate reduced coagulability of the blood in the presence of heparin in a simulated condition of a portion of a blood pump, where coagulation parameters include clotting initiation time (“R Time”) and clot formation time (“K Time”) in FIG. 6A; and clotting rates (a angle) in FIG. 6B;

FIG. 6C illustrates a TEG-ACT data illustrating synergistic influence on clotting with the presence of heparin and bicarbonate;

FIG. 7 illustrates a kit according to some embodiments;

FIG. 8 is a flowchart of an embodiment of a method;

FIG. 9 is an illustration of an embodiment of a system; and

FIG. 10 is a schematic illustration of a medical device according to some embodiments.

### DETAILED DESCRIPTION

Blood pumps are deployed in patients that require critical and life-saving care. Consequently, it is important to remediate any aspect of the device that might adversely affect pump operation. For example, in some pump designs, a purge fluid may be deployed to keep blood from entering the pump mechanism and to mitigate the effects of blood on the pump mechanisms. Typically, the purge fluid includes an anticoagulant such as heparin (e.g., the sodium salt of heparin). Without wishing to be bound by theory, heparin is thought to keep the blood from coagulating in the gap between pump components such as an impeller shaft and the housing. Heparin is a commonly used anticoagulant typically administered in controlled dosages.

In some instances, however, doctors may not want heparin to be administered to the patient's blood via the purge fluid. For example, administration of heparin during any sort of surgical procedure may be counterproductive as it prevents the coagulation of blood and, thus, healing or hemostasis. Also, the amount of heparin administered to the patient's blood along with heparin in the purge fluid is difficult to control for various reasons. In some instances, the amount of heparin may be more than what is desired by the doctors, and the amount of heparin administered to the patient may be difficult to precisely controlled. Accordingly, doctors may prefer to supply heparin to the patient separate from the operation of the blood pump, if needed (and then only in the amount needed). Furthermore, some patients are heparin-intolerant because they are susceptible to heparin-induced thrombocytopenia (HIT). So, a heparin-containing purge is not at all suitable for these patients. Also, salts of heparin can cause unwanted wear on pump bearings that are made of metal. Accordingly, there is a need for an intravascular blood pump which can run, if desired, with a purge fluid that contains no or at least a reduced amount of heparin.

It has been found that a particular fluid, by itself or in combination with heparin (e.g., systemic heparin treatment), can provide substantial benefit if present in specific gaps within an intravascular blood pump.

As disclosed herein, the inventors have recognized the benefit of a purge solution that contains sodium bicarbonate. The inventors have also recognized the benefit of using such a purge solution with systemically anticoagulated blood. For example, as described herein, the inventors have appreciated a synergistic or supportive role of sodium bicarbonate in the purge when used with system anticoagulation as part of an overall anticoagulation strategy. In some embodiments, sodium bicarbonate purge fluid may provide environmental conditions that delay initiation of coagulation, and suppress fibrin formation. This, in turn, may result in an overall decreased rate of clot formation where purge fluid and systemically anticoagulated blood mix in the axial gap.

In some embodiments, the purge fluid may include aqueous dextrose. For example, in some embodiments, the purge fluid may include a 5% dextrose in water solution. In some embodiments, the purge fluid may include 12.5 mEq/L to 100 mEq/L of sodium bicarbonate. In some embodiments, the purge fluid may include 25 mEq/L to 50 mEq/L of sodium bicarbonate.

In some embodiments, the purge fluid may also include a pharmaceutical therapeutic or prophylactic agent. In some embodiments, the pharmaceutical therapeutic or prophylactic agent may be an anticoagulant (such as heparin). In some embodiments, the pharmaceutical therapeutic or prophylactic agent may include a one or more small molecule drugs, which may be used for treating a patient, such as blood thinning agents, anti-inflammatory agents, etc.

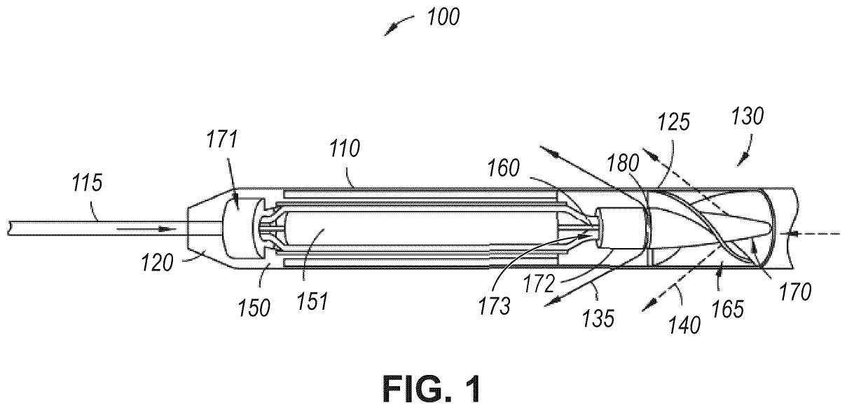

Turning now to the figures, FIG. 1 shows a pump 100 which possesses a tubular member having a drive section 110 and a pump section 130, a catheter 115 attached to a proximal end 120 of the drive section 110 (e.g., the end of the drive section closer to the doctor or “rear end” of the drive section) and having lines extending therethrough for the power supply to the drive section 110, and the pump section 130 fastened at the distal end 125 of the drive section. The drive section 110 may include a motor housing 150 having an electric motor 151 disposed therein, with the motor shaft 160 of the electric motor distally protruding out of the drive section 110 and into the pump section 130. The pump section 130 in turn may include a tubular pump housing 165 having an impeller 170 rotating therein which is seated on the end of the motor shaft 160 protruding out of the motor housing 150. The motor shaft 160 may be mounted in the motor housing in two bearings 171, 172 which are maximally removed from each other in order to guarantee a true, exactly centered guidance of the impeller 170 within the motor housing 150. Different bearing types may be used in different pump designs. As illustrated in FIG. 1, bearing 171 may include a radial ball bearing and bearing 172 may include an axial-radial sliding bearing. As illustrated in FIG. 1, blood 140 may exits the outflow cage of the pump housing 165. Blood that would otherwise enter into the motor housing 150 may be furthermore counteracted by a purge fluid 135 being passed through the motor housing and the impeller-side shaft seal bearing. Accordingly, the purge fluid may pass through the gap of the impeller-side radial sliding bearing so as to prevent blood from entering into the housing. This is done by having a purge fluid pressure that is higher than the pressure present in the blood.

As illustrated in FIG. 1, the purge fluid 135 may fill the motor housing 150 of the pump to form a lubricating film in the bearings 171, 172 of the pump. As described in U.S. Patent Publication No. 2015/0051436, for example, the purge fluid 135 may form a lubricating film in a bearing gap 180 of the axial slide bearing of a pump. Purge fluids are described as being fed through a purge-fluid feed line and flowing through the radial bearing gap 173 located at the distal end of the motor housing 150 and then also flowing through the bearing gap 180 of the axial sliding bearing. The purge fluids fed in this manner may be responsible for hemo-dilution and reduce blood retention time under the impeller 170.

In some embodiments, to ensure that the purge fluid 135 reaches the distal radial bearing 172 at a pressure higher than the blood pressure present, there is provided, in at least one of the surfaces forming the bearing gap of the axial sliding bearing, a channel which penetrates the bearing gap 180 from radially outward to radially inward, so that the purge fluid can flow through this channel to the distal radial bearing. This channel need not necessarily lie in a bearing-gap surface, but can also be realized as a separate channel or as a bore. However, in some embodiments, providing the channel in one of the bearing-gap surfaces may have the advantage that the lubricating film in the bearing gap heats up less, because a part of the lubricating film is continually being replaced by purge fluid flowing in later. In some embodiments, the channel is located in the stationary bearing-gap surface in order to minimize the radial conveying capacity.

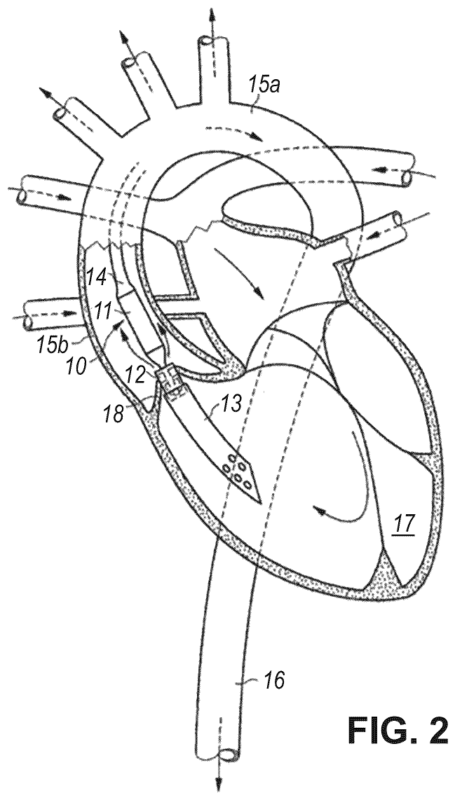

FIG. 2 illustrates the employment of a blood pump for supporting, in this particular example, the left ventricle. As shown in this figure, the blood pump may include a catheter 14 and a pumping device 10 attached to the catheter 14. The pumping device 10 may include a motor section 11 and a pump section 12 which are disposed coaxially one behind the other and result in a rod-shaped construction form. The pump section 12 may include an extension in the form of a flexible suction hose 13, a tubular member often referred to as “cannula.” An impeller may be provided in the pump section 12 to cause blood flow from a blood flow inlet to a blood flow outlet, and rotation of the impeller is caused by an electric motor disposed in the motor section 11. The blood pump may be placed such that it lies primarily in the ascending aorta 15b leading to the aortic arch 15a. The aortic valve 18 comes to lie, in the closed state, against the outer side of the pump section 12 or its suction hose 13 that lies substantially in the left ventricle 17. The blood pump with the suction hose 13 in front may be advanced into the represented position by advancing the catheter 14, optionally employing a guide wire. In so doing, the suction hose 13 may pass the aortic valve 18 retrograde, so the blood is sucked in through the suction hose 13 and pumped into the aorta 16.

As will be appreciated, the use of the blood pump need not be restricted to the application represented in FIG. 2, which merely involves a typical example of application. Thus, the pump can also be inserted through other peripheral vessels, such as the subclavian artery. Alternatively, reverse applications for the right ventricle may be envisioned. As will be further appreciated, other suitable pump arrangements may be used in other embodiments.

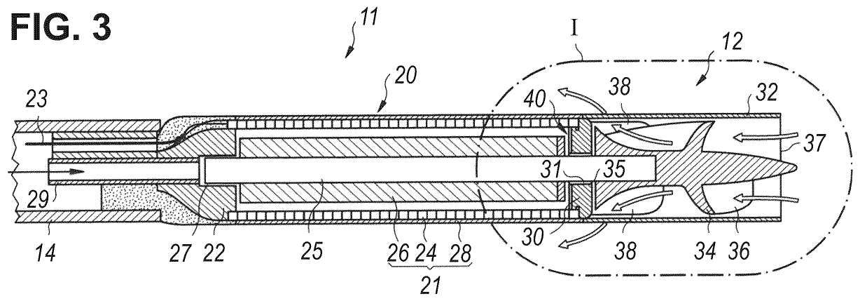

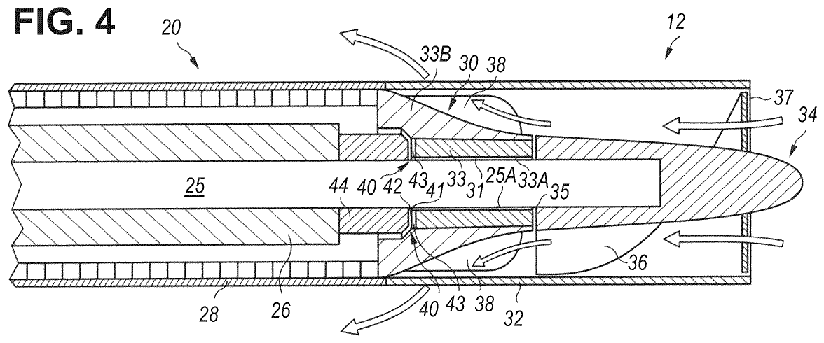

FIG. 3 shows an exemplary embodiment of a blood pump, such as that as described in US Patent Publication No. 2015/0051436 A1, which likewise may be suitable for use in the context of the present disclosure, except that the encircled front end marked with “I” may be modified, such modification being shown in FIG. 4. Accordingly, the motor section 11 may have an elongated housing 20 in which an electric motor 21 may be housed. A stator 24 of the electric motor 21 may have, in the usual way, numerous circumferentially distributed windings as well as a magnetic return path 28 in the longitudinal direction. The magnetic return path 28 may form an outer cylindrical sleeve of the elongate housing 20. The stator 24 may surround a rotor 26 connected to the motor shaft 25 and consisting of permanent magnets magnetized in the active direction. The motor shaft 25 may extend over the entire length of the motor housing 20 and protrude distally out of the latter through an opening 35. There, may carry an impeller 34 with pump vanes 36 projecting therefrom, which may rotate within a tubular pump housing 32 which may be firmly connected to the motor housing 20.

The proximal end of the motor housing 20 may have the flexible catheter 14 sealingly attached thereto. Through the catheter 14, there may extend electrical cables 23 for power supply to and control of the electric motor 21. In addition, a purge fluid line 29 may extend through the catheter 14 and penetrate a proximal end wall 22 of the motor housing 20. Purge fluid may be fed through the purge fluid line 29 into the interior of the motor housing 20 and exit through the end wall 30 at the distal end of the motor housing 20. The purging pressure may be chosen such that it is higher than the blood pressure present, in order to thereby prevent blood from penetrating into the motor housing. For example, in some embodiments, the purge pressure may be between 300 and 1400 mmHg depending on the case of application.

In some embodiments, the same purged seal may be combined with a pump that is driven by a flexible drive shaft and a remote motor.

Upon a rotation of the impeller 34, blood may be sucked in through the distal opening 37 of the pump housing 32 and conveyed backward within the pump housing 32 in the axial direction. Through radial outlet openings 38 in the pump housing 32, the blood flows out of the pump section 12 and further along the motor housing 20. In some embodiments, this ensures that the heat produced in the motor may be carried off. It also may be possible to operate the pump section with the reverse conveying direction, with blood being sucked in along the motor housing 20 and exiting from the distal opening 37 of the pump housing 32.

The motor shaft 25 may be mounted in radial bearings 27, 31 at the proximal end of the motor housing 20, on the one hand, and at the distal end of the motor housing 20, on the other hand. The radial bearings, in particular the radial bearing 31 in the opening 35 at the distal end of the motor housing, are configured as sliding bearings. Furthermore, the motor shaft 25 also may be mounted axially in the motor housing 20, the axial bearing 40 likewise being configured as a sliding bearing. The axial sliding bearing 40 serves for taking up axial forces of the motor shaft 25 which act in the distal direction when the impeller 34 conveys blood from distal to proximal. Should the blood pump be used for conveying blood also or only in the reverse direction, a corresponding axial sliding bearing 40 may (also or only) be provided at the proximal end of the motor housing 20 in a corresponding manner.

FIG. 4 shows the portion marked with “I” in FIG. 3 in greater detail, yet structurally modified according to an embodiment of present disclosure. There can be seen in particular the radial sliding bearing 31 and the axial sliding bearing 40. The bearing gap of the radial sliding bearing 31 is formed, on the one hand, by the circumferential surface 25A of the motor shaft 25 and, on the other hand, by the surface 33A of a through bore in a bushing or sleeve 33 of the end wall 30 of the motor housing 20 defining an outer gap diameter of about 1 mm, but the outer gap diameter may also be larger than this. In one example, the bearing gap of the radial sliding bearing 31 has a gap width of 2 μm or less not only at the front end or impeller-side of the gap but over the entire length thereof. In some embodiments, the gap width is between 1 μm and 2 μm. The length of the bearing gap may range from 1 mm to 2 mm, preferably from 1.3 mm to 1.7 mm, e.g., 1.5 mm. The surfaces forming the gap of the radial sliding bearing 31 may have a surface roughness of 0.1 μm or less. These dimensions will vary with the type of pump and are presented by way of example and not by way of limitation.

The bearing gap of the axial sliding bearing 40 may be formed, on the one hand, by the axially interior surface 41 of the end wall 30 and a surface 42 opposing it. This opposing surface 42 may be part of a ceramic disk 44 which is seated on the motor shaft 25 distally of the rotor 26 and rotates with the rotor 26. A channel 43 in the bearing-gap surface 41 of the end wall 30 ensures that purge fluid can flow through between the bearing-gap surfaces 41 and 42 of the axial sliding bearing 40 to the radial sliding bearing 31 and exit from the motor housing 20 distally. The axial sliding bearing 40 represented in FIG. 3 is a normal sliding bearing. Unlike the representation, the axial gap of the axial sliding bearing 40 is very small, being a few microns (e.g., 10 μm or less)

Instead of the axial sliding bearing 40 and radial sliding bearing 31, there can also be realized a combined radial-axial sliding bearing 40 having a concave bearing shell in which a convex bearing surface runs. Such a variant is represented in FIG. 4 by a spherical sliding bearing 40. The bearing-gap surface 41 may be of spherically concave design, and the opposing bearing-gap surface 42 is of corresponding spherically convex design. The channel 43 again may lie in the stationary bearing-gap surface 41 of the end wall 30. Alternatively, the stationary bearing-gap surface 41 of the end wall 30 can be of convex configuration and the opposing bearing-gap surface 42 of concave configuration. The surfaces 42, 43 can also be conical instead of spherical. In some embodiments, a corresponding radial-axial sliding bearing is provided on both sides of the motor housing 20 in order not to permit any radial offset upon axial travel of the shaft 25. The advantage of a combined axial-radial sliding bearing may lie in the higher loading capacity.

During operation, the blood pump may be attached to a purge-fluid source, and fluid passes into the motor housing through the purge-fluid line. The purge fluid then flows through the axial sliding bearing and further through the distal radial bearing. In the axial sliding bearing the purge fluid may form the lubricating film in the bearing gap. The pressure at which the purge fluid flows through the motor housing has an adverse effect, however, on the width of the bearing gap. Specifically, higher purge-fluid pressure may require a smaller bearing-gap width which results in a thinner lubricating film between the sliding surfaces. The thinner the lubricating film is, the greater the motor current that is required for driving the electric motor to overcome the frictional forces. This complicates the control of the blood pump, because the current conveying volume is normally established by stored characteristic curves solely on the basis of the motor current and the rotational speed (both known quantities). When the purge-fluid pressure also affects the motor current, this factor also has to be taken into consideration. In view of the fact that the same blood-pump type can be operated for a great variety of applications with different purge-fluid pressures between 300 and 1400 mmHg, it is important to avoid a dependence of motor current on purge-fluid pressure.

Such dependence is avoided when a purge fluid having a viscosity that is considerably higher than the viscosity of water (q=0.75 mPas at 37° C.) is selected. The viscosity of the purge fluid may be controlled by the concentration of dextrose in the purge fluid. Aqueous solutions of dextrose are widely administered to patients for a variety of reasons. The amount of dextrose in the aqueous solution may be between about 5% to about 50%. In one embodiment, the purge fluid may contain 5% dextrose in water (D5W, i.e., 278 mmol/liter). The viscosity can be increased by including solutions with a higher concentration of dextrose in water (e.g., D20W, D40W, etc.). When a highly viscous purge fluid is used, the fluid film may be maintained even at high pressures and the friction of the axial sliding bearing is accordingly independent of the purge-fluid pressure. In some embodiments, the axial sliding bearing can be configured as a simple sliding bearing, and does not have to be configured as a hydrodynamic sliding bearing, when a purge fluid having a viscosity at 37° C. that is about 1.2 mPas or higher.

In some embodiments, the pump impeller may induce shear stress on the blood passing through the pump. Shear stress may be induced in the gap between the impeller and the outer face of the ceramic bearing and between the impeller shaft and the inner race of the bearing (e.g., ceramic bearings, ball bearings, etc.). Due to the shear stresses to which the blood is subjected, blood proteins denature and polymerize as the blood passes through the pump. The deposition of the denatured and agglomerated protein causes activation of the clotting cascade, which, in turn, may cause the build-up of bio-deposits on the pump mechanisms (e.g., the impeller, the outflow cage, etc.). Small gaps between components (i.e., purge gaps) are particularly vulnerable to blockage by bio-deposits. The bio-deposit build-up will cause the motor current needed to operate the pump to increase. The increased motor current or bio-deposits can degrade pump performance or even cause a pump stop.

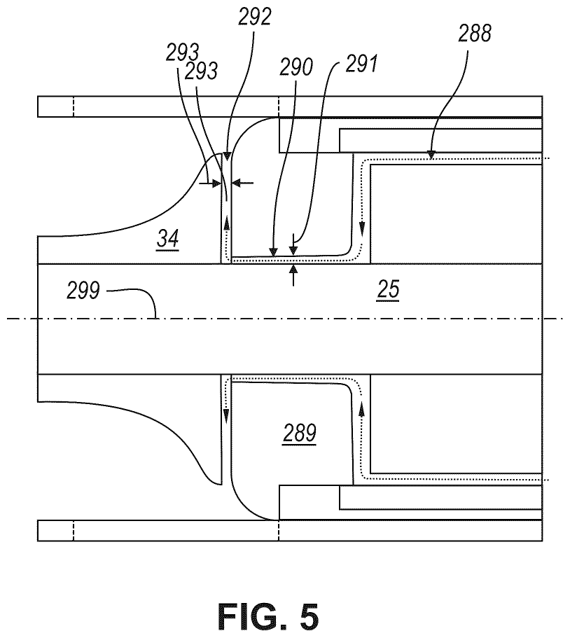

FIG. 5 illustrates another embodiment of purge fluid flowing through purge gaps. As shown in this view, the pump may include first gap 290 and second gap 292. In some embodiments, the first gap 290 may include a small radial gap 291 between an outer surface of a shaft 25 and an inner surface of a sleeve bearing 289, which may control the purge flow rate as the purge fluid 288 flows through the purge gaps. In some embodiments, the small radial gap 291 may extend in a radial direction between 4 μm and 9 μm, such as between 5 and 6 μm for some pumps and between 7 and 8 μm for other pumps. In some embodiments, the first gap may largely contain purge fluid. In some embodiments, a concentration of the purge fluid within a fluid filling the volume of space defined by the first gap may be at least 50%, at least 60%, at least 70%, at least 80%, at least 90%, at least 95%, or at least 99% of all fluid in the first gap. In some embodiments, the first gap is entirely filled with purge fluid. In some embodiments, due to flow co-mixing, some blood components may potentially reach the distal end of this gap. Due to the small dimensions, heat, and high shear, the biological material buildup in this location may include a denatured protein, which may result in rising purge pressures and increased friction leading to high motor current. In some embodiments, the most likely mechanism of action for bicarbonate with an illustrative pump (e.g., the Impella® pump) in the first gap is to neutralize the acidic pH of the D5 carrier solution to reduce denaturation and adsorption of blood proteins in the radial gap.

In some embodiments, the second gap 292 may include an axial gap 293 between the impeller 34 and a sleeve bearing 289. In some embodiments, the axial gap 293 may extend in an axial direction (e.g., a distance parallel to axis 299 of the impeller shaft) of between about 90 and 110 μm, such as about 100 μm. In some embodiments, the second gap may be where purge fluid and blood initially mix. Purge fluid may flow along a back side of the appeal, with system blood being pulled into the gap setting up a clockwise flow pattern in this micro-environment. In some embodiments, a volume of blood pulled into the second gap may be between about 0.75 and 1.1 cubic mm, depending upon the pump. In some embodiments, the purge fluid and blood do not fully mix in the axial gap. As will be appreciated, the mixing ratio of blood to purge fluid in the second gap may vary as the purge flow varies. For example, at higher purge flows (e.g., 30 ml/hr), the ratio may be about 60% blood based, while at lower purge flows (e.g., about 0 ml/hr), the ratio may approach 100% blood. That is, in some embodiments, depending on the flow rate of the purge fluid, a concentration of the purge fluid within a fluid filling the volume of space defined by the second gap may at least 40%, or may be not more than 40%, not more than 30%, not more than 20%, or not more than 10% of all fluid in the first gap.

As noted above, to mitigate the adverse effects of shear on the blood that flows through the pump, the purge fluids used in purged blood pumps typically include the anticoagulant heparin (e.g., 50 units/ml) in 5%-Dextrose (D5W). In some embodiments, heparin is provided in the purge fluid to prevent the formation of shear-induced bio-material or bio-deposits, and the resulting undesirable deposition/accumulation of biological material in the pump, such as between the impeller shaft and the inner race of the bearings at high shear areas. However, as noted above, there may be challenges associated with adding heparin to the purge fluid. Specifically, heparin: a) may make systematic anticoagulant management complex (i.e., there is a need to consider the heparin dose that the patient is receiving via the purge fluid); b) heparin, as an anticoagulant, may increase a patient's propensity to bleed; c) may make it more difficult to control bleeding in post-operative patients, especially when surgical devices are used on such patients; and d) cannot be used for heparin-induced thrombocytopenia (HIT) patients. Also, heparin may also be administered systemically to some patients, making it difficult to regulate the administration of two source of heparin

As described herein, the dextrose concentration may determine the viscosity of the purge fluid and hence affects the purge flow rate. Purge fluids with lower dextrose concentrations are less viscous and flow more quickly with less pressure through the purge system. Purge fluids with higher dextrose concentrations (more viscous) result in a lower purge flow rate and require a greater purge pressure. For example, a reduction in dextrose concentration from 20% to 5% may results in an approximately 30% to 40% increase in purge flow rates.

Purge flow rates are typically in the range of about 2 mL/hour to about 30 mL/hour. This results in a purge pressure of about 1100 mmHg to about 300 mmHg. Typical purge flows for the blood pumps described herein, e.g., Impella CP, Impella 2.5, Impella 5.0/LD, and RP, are about 5 mL/hour to about 20 mL/hour. These pumps all have a ball-bearing rotor/stator system with similar tolerances leading to similar purge operation ranges. Typical purge flows for the Impella 5.5 are about 2 to about 10 mL/hour. This lower flow rate results from the deployment of a ceramic bearing rotor/stator system designed with a reduced purge gap (radial) to reduce or eliminate the amount of heparin delivered to the patient. For surgical patients, surgeons prefer not to administer heparin in the first few days after surgery. For these patients, then, purge fluids that contain no heparin are preferred.

According, a purge fluid/purge fluid additive that can mitigate problems in pump performance caused by the pump operation is still needed. It has been observed that the denatured proteins become prone to agglomeration as protein unfolding exposes hydrophobic regions of a protein. This causes unwanted bio-deposition. Absent denaturing and agglomeration, the hydrophobic segments are shielded, and protein molecules are repulsed due to the electrostatically charged groups of the protein.

Soluble calcium ions are known to mediate coagulation. The human serum albumin in blood controls the calcium ions. At higher pH values, the albumin more strongly retains the calcium ions. This mechanism reduces the effective concentration of calcium available for coagulation. Therefore, providing an additive to the purge fluid that elevates the pH of the purge fluid will reduce the amount of calcium that will support coagulation in the high stress areas. Described herein are relatively high pH buffers for use in a purge fluid that will elevate blood pH and therefore mitigate clotting.

Contemplated herein are pH-controlling and buffering agents that are added to the purge fluid that avoid the problems of heparin but meet the other objectives of the purge fluid (bio deposit mitigation; bearing wear reduction; higher pressure than blood pressure, etc.). One example of a suitable pH-controlling and buffering agent is sodium bicarbonate. However, pH-controlling and buffering agents other than sodium bicarbonate are also contemplated. Those pH controlling and buffering agents, include, for example, salts of small organic acids, such as citrate, lactate, gluconate, acetate, pyruvate, etc. In one example, the pH of sodium bicarbonate is about 7.4 to about 9.1. In one example, the pH of the purge fluid with bicarbonate is about 8.4. Other ranges, including but not limited to about 7.5 to about 9.1, 7.6 to about 9.1, 7.7 to about 9.1, 7.8 to about 9.1, 7.9 to about 9.1, 8.0 to about 9.1, 8.1 to about 9.1, 8.2 to about 9.1, 8.3 to about 9.1, 8.4 to about 9.1, 8.5 to about 9.1, 8.6 to about 9.1, 8.7 to about 9.1, 8.8 to about 9.1, 8.9 to about 9.1 and 9.0 to about 9.1 are contemplated. The pH of blood is about 7.3 to about 7.4. Adding sodium bicarbonate to the purge fluid will elevate the pH of the blood that comes into contact with the purge fluid. The elevated pH in those purge gaps (e.g., first and second gaps 290, 292, as shown in FIG. 5) will reduce bio deposits that result from blood coagulation caused by the high shear pump environment. Due to this effect, the presence of sodium bicarbonate, even if coagulation proceeds towards formation of individual fibrin molecules, may reduce the formation of insoluble bio deposits.

In one embodiment, adding a solution containing bicarbonate mixed with a dextrose solution such as dextrose 5% in water (D5W), dextrose 20% in water (D20W) dextrose 40% in water (D40W), etc. to blood may increase the local pH of the blood at the gaps (higher shear area) and prevents the agglomeration of the protein by increasing the electrostatic charge of the serum protein, and therefore reduces formation of bio-deposition. The amount of bicarbonate in the solution of bicarbonate mixed with the dextrose solution may be about about 1.5 milliequivalents per liter (mEq/L) to about 50 mEq/L. In some embodiments, the amount of sodium bicarbonate may be 12.5 mEq/L to 100 mEq/L, such as between 25 mEq/L and 50 mEq/L. In such embodiments, the purge solution may include no heparin. In such embodiments, the pH in the gaps may be configured to maintain an optimal presentation of positively charged key amino acids of Heparin targets.

Described herein are systems and methods that use sodium bicarbonate as an alternative to heparin in a purge fluid used to maintain the patency of a purge system for a blood pump, such as at the first and second gaps. The sodium bicarbonate may be deployed in the purge system and not used for systemic anticoagulation of the patient in which the pump is deployed. Instead, systemic anticoagulation using heparin, bivalirudin, and/or argatroban may be used to prevent thromboembolic events, even when sodium bicarbonate is used in the purge. As will be appreciated, other suitable anticoagulation agents may be used in other embodiments.

According to some embodiments, systemic anticoagulation (e.g., using heparin) with a sodium bicarbonate purge liquid may limit biomaterial deposition and the risk of embolization. For example, the sodium bicarbonate purge fluid may maintain slightly higher neutral pH in mixtures of blood and purge fluid at the purge gaps (e.g., first and second gaps 290 and 292). This may result in environmental conditions that 1) support protein stability in the radial gap to maintain purge flow patency, and 2) delay initiation of coagulation, suppress fibrin formation, with an overall decreased rate of clot formation where purge fluid and systemically anticoagulated blood mix in the second gap (e.g., the axial gap).

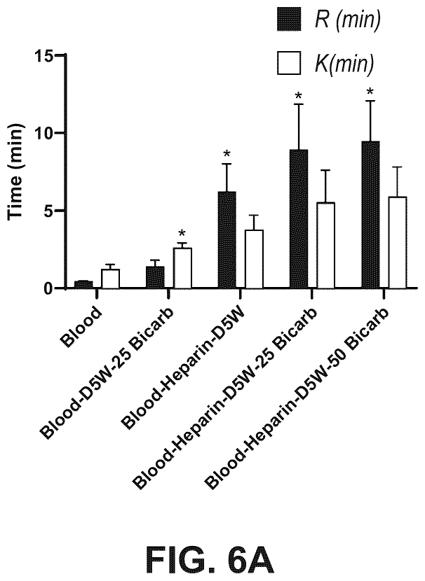

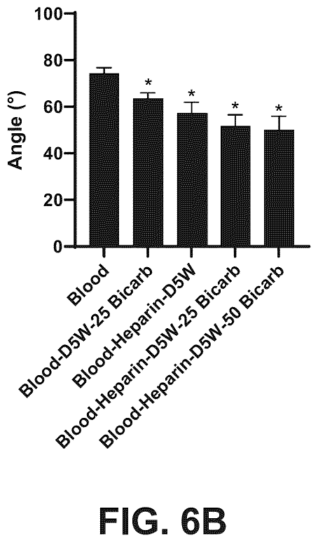

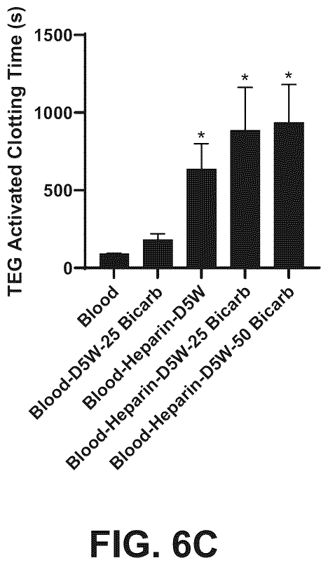

As shown in FIGS. 6A, 6B, and 6C, the effects of sodium bicarbonate in the purge gap environment were simulated by conducting in vitro coagulability experiments using Thromboelastography (TEG). As shown in the figures, systematically anticoagulated blood in the presence of sodium bicarbonate may show delayed initiation of clot formation and reduced rate of clot formation with systemically anticoagulated, as compared to baseline blood (R and K (6A), angle parameters (6B), and TEG-ACT (6C)). In such embodiments, the effects of sodium bicarbonate in the purge gap environment was simulated with 60% blood and 40% purge fluid (e.g., to represent purge fluid concentrations in a specific purge gap environment at the high purge flow rate of 30 mL/hr). It will be understood that even if the purge fluid concentration may be 40% in the local micro-environment, the overall concentration of the purge fluid in a patient's bloodstream will be substantially lower. For example, the total dose of bicarbonate delivered to a patient may be 12 mEq (or less) over 8 hours. In some embodiments, in the case of purge fluid containing sodium bicarbonate mixed with blood, that pH in the Impella gaps may maintain an optimal presentation pattern of positively charged key amino acids of Heparin targets and that the conditions for systemic heparin action are optimal.

As shown in FIG. 6C, TEG-ACT was used to assess the interaction of purge fluid mixing with systemically heparinized blood in gap 2 (e.g., the axial gap). Again, the simulation was performed with 60% blood and 40% purge fluid (e.g., to represent purge fluid at the high purge fluid flow rate of 30 mL/hr). As shown FIG. 6C, a synergic impact on blood coagulability was observed in the presence of bicarbonate and heparin.

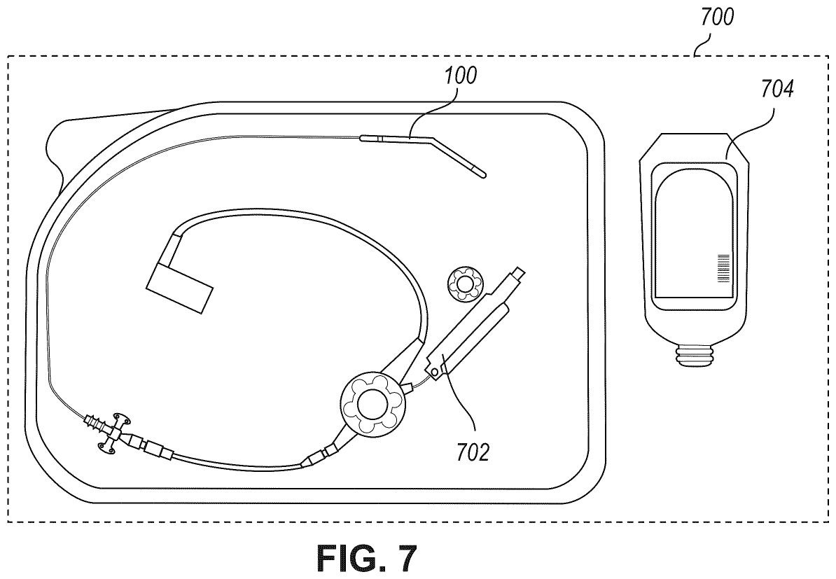

FIG. 7 illustrates a kit according to some embodiments of the present disclosure. As shown in this view, in some embodiments, the kit 700 includes a pump 100, such as a pump including one or more feature described herein. In some embodiments, the pump may include an inlet area, a cannula, one or more sensors, an outlet area, a motor housing, and a catheter. In some embodiments, as shown in this view, the kit may include a purge cassette 702 configured to deliver a purge fluid to and through the catheter and to the pump, such as to prevent blood from entering the motor. As will be appreciated, the purge cassette is configured to be attached to a purge fluid, such as to the purge bag 704. In some embodiments, the purge bag includes a premixed solution with sodium bicarbonate. For example, in some embodiments, the purge fluid may include a 5% dextrose in water solution with between 12.5 and 100 mEq/L, such as between 25 mEq/L and 50 mEq/L of sodium bicarbonate. In some embodiments, the amount of sodium bicarbonate is 25 mEq/L.

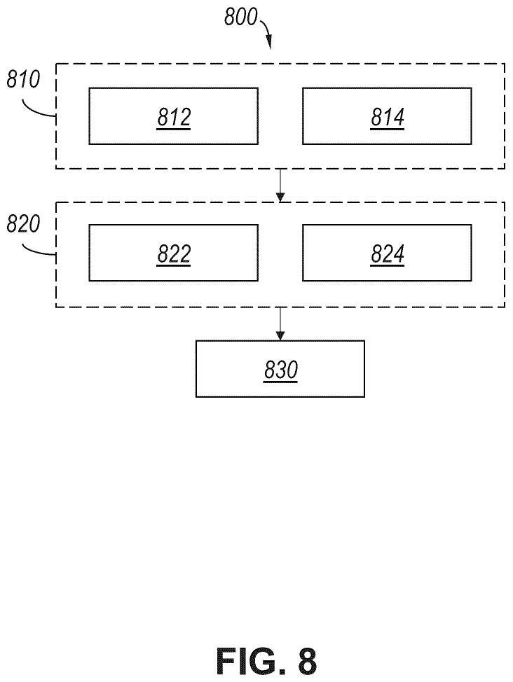

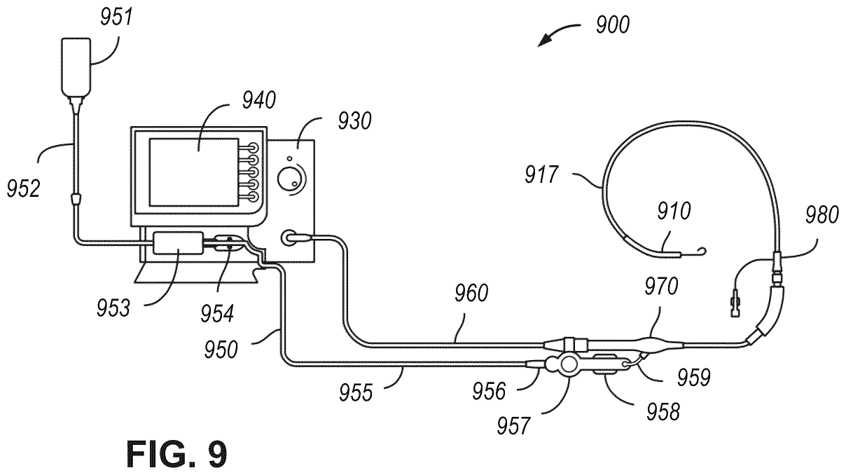

Referring to FIG. 8, a method may be provided. The method may include providing 810 the required components. This may include providing 812 a pump as disclosed herein, and may include providing 814 a purge fluid as described herein. Other components may be seen in FIG. 9.

Referring to FIG. 9, a blood pump assembly 900 may include a blood pump 910 fluidically connected to a container 951 (such as a purge bag) that contains a purging fluid as disclosed herein, through a purging device 953. The blood pump assembly 900 also may include a controller 930 (e.g., an Automated Impella Controller® from Abiomed, Inc., Danvers, MA), a display 940, a connector cable 960, a plug 970, and a repositioning unit 980. As shown, the controller 930 may include a display 940. Controller 930 may monitor and controls blood pump 910. During operation, purging device 953 may deliver a purge fluid as disclosed herein to blood pump 910 through a first line 950, 955 (e.g., a tube), through one or more components 956, 957, 958, 959 and through a catheter tube 917, such as to prevent blood from entering the motor (not shown) within a motor housing of the pump. Connector cable 960 may provide an electrical connection between blood pump 910 and controller 930. Plug 970 connects catheter tube 917, purging device 953, and connector cable 960. In some embodiments, plug 970 may include a memory for storing operating parameters in case the patient needs to be transferred to another controller. Repositioning unit 980 may be used to reposition blood pump 910. As shown in this view, the fluid line 950, 955 may be separate from the connector cable 960 having one or more electrical wires.

The method may include operating the pump 820, which may include rotating 822 the impeller of the pump. The method may also include flowing 824 the purge fluid into the disclosed gaps of the pump. This may be done by controlling the purge device 953 (which may include, e.g., a positive displacement pump). This may include flowing the fluid through a first gap between a bearing and an outer surface of a rotatable shaft coupled to the impeller, the bearing and gap being disposed within a lumen of a tubular member. In some embodiments, this may include flowing the fluid through a second gap after passing through the first gap, the second gap being between the bearing and a surface of the impeller facing the bearing.

In some embodiments, the method may include controlling 830 a flow rate of the fluid into the pump during operation. In some embodiments, this may include controlling the flow rate such that a concentration of the fluid comprising the bicarbonate within a volume of space defined by the first gap is at least 50% of all fluid in the first gap. In some embodiments, this may include controlling a flow rate of the fluid such that a concentration of the fluid comprising the bicarbonate within a volume of space defined by the second gap is no more than 40% of all fluid in the second gap.

In some embodiments, the controller may be configured to control the flow rate of the fluid into the pump based on a speed of the impeller. For example, at low impeller rotational velocities, the flow rate of the fluid may be relatively low, and as the impeller increases in speed, the flow rate of the fluid will automatically be increased to offset the increased pressure of blood attempting to enter the gap(s) of the pump. In some embodiments, this blood flow is controlled to provide a substantially constant ratio of blood-to-fluid containing bicarbonate within each gap. For example, if the flow rate is initially selected to provide an 80%/20% blood/fluid containing bicarbonate ratio, and then later, the impeller speed increases (or decreases) (which would cause that 80/20 ratio to change), the method may include controlling the flow rate of the fluid into the pump to increase (or decrease) accordingly to keep the ratios substantially equal. In some embodiments, this is done via a correlation based on empirical data. In some embodiments, the controller is configured to solve a predetermined function relating flow rate to, e.g., impeller speed.

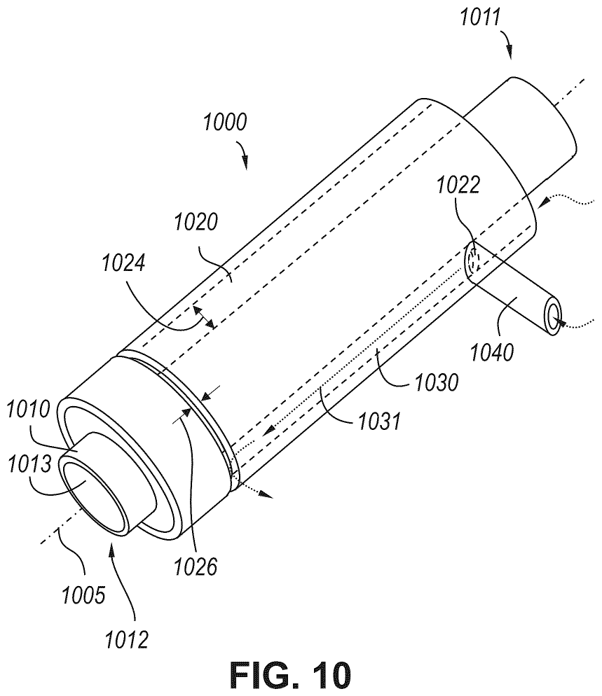

Although embodiments disclosed herein include affecting the micro-environment of purges gaps (e.g., axial and radial gaps) within a blood pump (e.g., a percutaneous blood pump) via a purge fluid containing sodium bicarbonate, it will be appreciated that such a sodium bicarbonate purge may be used with other components of a blood pump system, or even other medical devices (or other medical device components), having similar purge gaps. For example, in some embodiments, as shown in the schematic representation of FIG. 10, a sodium bicarbonate purge fluid 1030 may be used to purge an axial gap 1026 and a radial purge gap 1024 between a tubular body 1010 (such as a catheter) having a distal end 1012 and a proximal end 1011 (and may include a lumen 1013 extending therethrough, the lumen having a central axis 1005), and an outer sheath 1020 (e.g., an accessory sheath such as a sheath used for insertion and/or holding the pump in place in the patient) disposed around the tubular body. The purge fluid may be configured to flow in a direction 1031 (e.g., generally moving axially when in the radial purge gap, and radially in the axial gap) through the radial purge gap and the axial gap to purge those gaps. In an illustrative embodiment, this may be used to flush a sidearm 1040 of a sheath (which may be operably connected to the radial purge gap via, e.g., a port 1022 in the side of outer sheath 1020), such as to minimize clot formation. In some embodiments, as shown in FIG. 10, the axial gap may be located near a first end of the outer sheath. However, it will be appreciated that the axial gap may be located at any suitable portion along a length of the outer sheath, such as near the sidearm of the outer sheath.

In some embodiments, a maximum distance D1 between a first edge of an axial gap and an end of the outer sheath may be <10 cm. In some embodiments, D1<5 cm. In some embodiments, D1<4 cm. In some embodiments, D1<3 cm. In some embodiments, D1<2 cm. In some embodiments, D1<1 cm. In some embodiments, a maximum distance D2 between a first edge of the axial gap and a center of a port (e.g., port 1022) in the outer sheath may be <5 cm. In some embodiments, D2<4 cm. In some embodiments, D2<3 cm. In some embodiments, D2<2 cm. In some embodiments, D2<1 cm.

As will be appreciated, and as shown in this view, the axial and radial gaps may be formed between the outer diameter of the catheter and the inner diameter of the sheath. In some embodiments, as will be appreciated, the ratio of blood-to-fluid containing bicarbonate be higher than that experienced in the bearing gaps shown and described with respect (e.g., higher than 80%/20% and increasing to 100% blood). Although not shown, it will be appreciated, that a fluid line may be connected to the sheath such that the purge fluid may be transferred into the purge gaps. As with the above, a controller may be used to control the rate at which the purge fluid is delivered to the sheath.

While particular embodiments of this technology have been described, it will be evident to those skilled in the art that the present technology may be embodied in other specific forms without departing from the essential characteristics thereof. The present embodiments and examples are therefore to be considered in all respects as illustrative and not restrictive. It will further be understood that any reference herein to subject matter known in the field does not, unless the contrary indication appears, constitute an admission that such subject matter is commonly known by those skilled in the art to which the present technology relates.

1. A blood pump, comprising:
a tubular body having a distal end and a proximal end, and a lumen extending therethrough, the lumen having a central axis;
an impeller coupled to a flexible shaft disposed at least partially within the lumen; and
a bearing within the lumen, the bearing being disposed around a portion of the flexible shaft, and configured to form a first gap in a radial direction between an outer surface of the flexible shaft and an inner surface of the bearing, and to form a second gap in an axial direction is between the bearing and a surface of the impeller facing the bearing;
wherein the blood pump is configured to flow a fluid comprising a bicarbonate through the first gap and into the second gap.

2. The blood pump according to claim 1, wherein the bicarbonate is sodium bicarbonate.

3. The blood pump according to claim 2, wherein the fluid contains 12.5 mEq/L to 100 mEq/L of sodium bicarbonate.

4. The blood pump according to claim 1, wherein the fluid further comprises a pharmaceutical therapeutic or prophylactic agent.

5. The blood pump according to claim 4, wherein the pharmaceutical therapeutic or prophylactic agent is an anticoagulant.

6. The blood pump according to claim 1, wherein the fluid further comprises aqueous dextrose.

7. The blood pump according to claim 1, wherein a concentration of the fluid comprising the bicarbonate within a volume of space defined by the first gap is at least 50% of all fluid in the first gap.

8. The blood pump according to claim 7, wherein the first gap is filled with the fluid comprising the bicarbonate.

9. The blood pump according to claim 8, wherein a concentration of the fluid comprising the bicarbonate within a volume of space defined by the second gap is no more than 40% of all fluid in the second gap.

10. The blood pump according to claim 1, wherein the first gap is 4-9 μm.

11. The blood pump according to claim 1, wherein the second gap is 90-110 μm.

12. A system, comprising:
a pump comprising:
a tubular body having a distal end and a proximal end, and a lumen extending therethrough, the lumen having a central axis;
an impeller coupled to a flexible shaft disposed at least partially within the lumen;
a bearing within the lumen, the bearing being disposed around a portion of the flexible shaft, and configured to form a first gap in a radial direction between an outer surface of the flexible shaft and an inner surface of the bearing, and to form a second gap in an axial direction is between the bearing and a surface of the impeller facing the bearing;
wherein the pump is configured to flow a fluid comprising a bicarbonate through the first gap and into the second gap; and
a purge bag containing the fluid comprising the bicarbonate, the purge bag being operably coupled to the pump.

13-35. (canceled)

36. A medical device comprising:
a tubular body having a distal end and a proximal end, and a lumen extending therethrough, the lumen having a central axis;
an outer sheath disposed around the tubular body;
a first gap formed in a radial direction between an outer surface of the tubular body and the inner surface of the outer sheath;
a second gap formed in an axial direction between the tubular body and the outer sheath;
wherein a fluid comprising a bicarbonate is configured to flow through the first and second gaps to purge the first and second gaps.

37. The medical device according to claim 36, wherein the bicarbonate is sodium bicarbonate.

38. The medical device according to claim 37, wherein the fluid contains 12.5 mEq/L to 100 mEq/L of sodium bicarbonate.

39. The medical device according to claim 36, wherein the fluid further comprises a pharmaceutical therapeutic or prophylactic agent.

40-41. (canceled)

42. The medical device according to claim 36, wherein a concentration of the fluid comprising the bicarbonate within a volume of space defined by the first gap is at least 50% of all fluid in the first gap.

43. The medical device according to claim 42, wherein the first gap is filled with the fluid comprising the bicarbonate.

44. The medical device according to claim 43, wherein a concentration of the fluid comprising the bicarbonate within a volume of space defined by the second gap is no more than 40% of all fluid in the second gap.

45. The medical device according to claim 36, wherein the first gap is 4-9 μm.

46. The medical device according to claim 36, wherein the second gap is 90-110 μm.
